(19) United States
(12) Patent Application Publication
(10) Pub. No.: US 2012/0230921 A1
(43) Pub. Date: Sep. 13, 2012

(54) NITRIC OXIDE RELEASING PARTICLES FOR ORAL CARE APPLICATIONS

(76) Inventor: Nathan Stasko, Durham, NC (US)

(21) Appl. No.: 13/477,558

(22) Filed: May 22, 2012

Related U.S. Application Data
(63) Continuation of application No. 12/580,418, filed on Oct. 16, 2009.
(60) Provisional application No. 61/105,951, filed on Oct. 16, 2008.

(51) Int. Cl. A61K 8/58; A61Q 11/00

(52) U.S. Cl. 424/49

(57) ABSTRACT

The present invention provides an oral care composition comprising a nitric oxide releasing particle and an orally-acceptable carrier. The nitric oxide releasing particle comprises at least one nitric oxide donor. Another aspect of the present invention provides a device for oral care comprising a nitric oxide releasing particle, wherein the device is configured to expose a targeted site in an oral cavity of a subject to nitric oxide. The present invention also provides methods and uses of providing oral health benefits by using the oral care compositions or devices as described above.

### RELATED APPLICATIONS

This application is a continuation application of U.S. application Ser. No. 12/580,418; filed Oct. 16, 2009, which claims the benefit under 35 U.S.C. §119(e) of U.S. Provisional Patent Application Ser. No. 61/105,951; filed Oct. 16, 2008, the disclosures of which are incorporated herein by reference in their entireties.

### FIELD OF THE INVENTION

The present invention generally relates to nitric oxide releasing particles for oral care and other medical applications and uses.

### BACKGROUND OF THE INVENTION

In the last two decades, nitric oxide (NO) has been implicated in a number of bioregulatory processes including normal physiological control of blood pressure, macrophage destruction of foreign pathogens, and neurotransmission. Recent research has further demonstrated that nitric oxide possesses a broad-spectrum of antimicrobial activity and may be used as an alternative to conventional antibiotics for drug resistant bacteria. In addition, nitric oxide may also be used to alleviate inflammation and promote wound healing.

However, nitric oxide is a gas at ambient temperature and atmospheric pressure, and it has a short half-life in physiological milieu. Thus, it is relatively challenging to deliver nitric oxide in a controlled and targeted manner and use nitric oxide to treat bacterial infection and/or diseases. The application of nitric oxide has been relatively limited because of the absence of a controlled and targeted delivery method or material.

The use of nitric oxide in oral care has been challenging. For example, the nitric oxide delivery systems for treating oral health problems like microbial plaque biofilms, gingivitis, and periodontal disease are required to maintain the stability in the environment of variable pH of saliva and the temperature in the mouth. Moreover, the nitric oxide system may also be required to release nitric oxide spontaneously via non-enzymatic mechanisms and in a controlled and targeted manner. In addition, the materials that carry nitric oxide should be compatible with other oral care products and it should be non-interactive after releasing nitric oxide.

Therefore, it is desired to deliver nitric oxide in a controlled and targeted manner, particularly for oral care and other medical applications and uses.

### SUMMARY OF THE INVENTION

One aspect of the present invention provides an oral care composition comprising a nitric oxide releasing particle and an orally-acceptable carrier. In some embodiments, the nitric oxide releasing particle comprises at least one nitric oxide donor. In another embodiment, the nitric oxide releasing particle comprises at least one nitric oxide donor and a co-condensed silica network.

In some embodiments, the nitric oxide donor is diazeniumdiolate and the nitric oxide donor is formed from an aminoalkoxysilane by a pre-charging method such that diazeniumdiolated aminoalkoxysilane is formed, and then the co-condensed silica network is synthesized from the condensation of a silane mixture comprising an alkoxysilane and the diazeniumdiolated aminoalkoxysilane to form a nitric oxide donor modified co-condensed silica network. In some embodiments, the nitric oxide donor is a diazeniumdiolate. In some embodiments, the orally acceptable carrier is a toothpaste, a mouth wash or a dental floss.

Another aspect of the present invention provides a device for oral care comprising a nitric oxide releasing particle, wherein the device is configured to expose a targeted site in an oral cavity of a subject to nitric oxide, and the nitric oxide releasing particle comprises at least one nitric oxide donor. In some embodiments, the nitric oxide releasing particle further comprises a co-condensed silica network.

In some embodiments, the device is in a form of a mouthguard, a dental sealant, a medical device, a dental implant, a tray, a strip, a syringe, a cover, a pad/patch, a film, a sponge, a cream, a fiber, or a gel that is adapted to be applied on the targeted site.

In some embodiments, for the device for oral care, the co-condensed silica network further comprises at least one crosslinkable functional moiety of formula (R₁)ₓ(R₂)_{y}SiR₃, wherein: R₁ and R₂ is each independently C₁₋₅ alkyl or C₁₋₅ alkoxyl; X and Y is each independently 0, 1, 2, or 3; and X+Y equal to 3; and R₃ is a crosslinkable functional group.

Another aspect of the present invention discloses a method of providing one or more oral health benefits to a subject comprising contacting an effective amount of an oral care composition in an oral cavity of the subject. The oral care composition comprises nitric oxide releasing particle and an orally-acceptable carrier.

One aspect of the present invention provides a method of providing one or more oral health benefits to a subject by exposing a targeted site in an oral cavity of the subject to a device comprising nitric oxide releasing particles. The device is configured to release a therapeutically effective amount of nitric oxide. In some embodiments, the amount and rate of the release of nitric oxide is regulated by adjusting pH of the oral cavity or light exposed to the nitric oxide particles.

Objects of the present invention will be appreciated by those of ordinary skill in the art from reading the detailed description of the preferred embodiments which follow, such description being merely illustrative of the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 schematically demonstrates the preparation of nitric oxide loaded silica.

FIG. 2 schematically demonstrates the preparation of multifunctional nitric oxide loaded silica.

FIG. 3 schematically demonstrates the preparation of antimicrobial nitric oxide releasing particle.

FIG. 4 schematically demonstrates methods of incorporating nitric oxide releasing silica into a polymeric matrix.

FIG. 5 graphically demonstrates the log reduction in CFU/mL of Streptococcus mutans, a gram positive bacterium commonly found in the oral cavity when exposed to nitric oxide releasing co-condensed silica particles at various doses.

### DETAILED DESCRIPTION

The foregoing and other aspects of the present invention will now be described in more detail with respect to the description and methodologies provided herein. It should be appreciated that the invention may be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, dose, time, temperature, and the like, is meant to encompass variations of 20%, 10%, 5%, 1%, 0.5%, or even 0.1% of the specified amount. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this invention belongs.

All patents, patent applications and publications referred to herein are incorporated by reference in their entirety. In case of a conflict in terminology, the present specification is controlling.

### Definitions

As used herein the term “alkyl” refers to C-₁-20 inclusive, linear (i.e.,“straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tent-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. Exemplary branched alkyl groups include, but are not limited to, isopropyl, isobutyl, tert-butyl. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₅ straight-chain alkyls. In other embodiments, “alkyl” refers, in particular, to C₁₋₅ branched-chain alkyls.

Alkyl groups may optionally be substituted (a “substituted alkyl”) with one or more alkyl group substituents, which may be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There may be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic substituent that may be a single aromatic ring, or multiple aromatic rings that are fused together, linked covalently, or linked to a common group, such as, but not limited to, a methylene or ethylene moiety. The common linking group also may be a carbonyl, as in benzophenone, or oxygen, as in diphenylether, or nitrogen, as in diphenylamine. The term “aryl” specifically encompasses heterocyclic aromatic compounds. The aromatic ring(s) may comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group may be optionally substituted (a “substituted aryl”) with one or more aryl group substituents, which may be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR¹R″, wherein R¹ and R″ may each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto. Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group may be optionally partially unsaturated. The cycloalkyl group also may be optionally substituted with an alkyl group substituent as defined herein, oxo, and/or alkylene. There may be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include, but are not limited to, cyclopentyl, cyclohexyl, and cycloheptyl. Multicyclic cycloalkyl rings include, but are not limited to, adamantyl, octahydronaphthyl, decalin, camphor, camphane, and noradamantyl.

“Alkoxyl” refers to an alkyl-O— group wherein alkyl is as previously described. The term “alkoxyl” as used herein may refer to, for example, methoxyl, ethoxyl, propoxyl, isopropoxyl, butoxyl, f-butoxyl, and pentoxyl. The term “oxyalkyl” may be used interchangeably with “alkoxyl”. In some embodiments, the alkoxyl has 1, 2, 3, 4, or 5 carbons.

“Aralkyl” refers to an aryl-alkyl-group wherein aryl and alkyl are as previously described, and included substituted aryl and substituted alkyl. Exemplary aralkyl groups include, but are not limited to, benzyl, phenylethyl, and naphthylmethyl.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group may be straight, branched or cyclic. The alkylene group also may be optionally unsaturated and/or substituted with one or more “alkyl group substituents.” There may be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include, but are not limited to, methylene (—CH₂—); ethylene (—CH₂—CH₂—); propylene (—(CH₂)₃—); cyclohexylene (—C₆H₁₀—); —CH═CH—CH═CH—; —CH═CH—CH₂—; wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (-0-CH₂—O—); and ethylenedioxyl (—O—(CH₂)₂—O—). An alkylene group may have about 2 to about 3 carbon atoms and may further have 6-20 carbons.

“Arylene” refers to a bivalent aryl group. An exemplary arylene is phenylene, which may have ring carbon atoms available for bonding in ortho, meta, or para positions with regard to each other, i.e., respectively. The arylene group may also be napthylene. The arylene group may be optionally substituted (a “substituted arylene”) with one or more “aryl group substituents” as defined herein, which may be the same or different.

“Aralkylene” refers to a bivalent group that contains both alkyl and aryl groups. For example, aralkylene groups may have two alkyl groups and an aryl group (i.e., -alkyl-aryl-alkyl-), one alkyl group and one aryl group (i.e., -alkyl-aryl-) or two aryl groups and one alkyl group (i.e., -aryl-alkyl-aryl-).

The term “amino” and “amine” refer to nitrogen-containing groups such as NR₃, NH₃, NHR₂, and NH₂R, wherein R may be alkyl, branched alkyl, cycloalkyl, aryl, alkylene, arylene, aralkylene. Thus, “amino” as used herein may refer to a primary amine, a secondary amine, or a tertiary amine. In some embodiments, one R of an amino group may be a cation stabilized diazeniumdiolate (i.e., NONO⁻X⁺).

The terms “cationic amine” and “quaternary amine” refer to an amino group having an additional (i.e., a fourth) group, for example a hydrogen or an alkyl group bonded to the nitrogen. Thus, cationic and quaternary amines carry a positive charge.

The term “alkylamine” refers to the -alkyl-NH₂ group.

The term “carbonyl” refers to the —(C═O)— group.

The term “carboxyl” refers to the —COOH group and the term “carboxylate” refers to an anion formed from a carboxyl group, i.e., —COO⁻.

The terms “halo”, “halide”, or “halogen” as used herein refer to fluoro, chloro, bromo, and iodo groups.

The term “hydroxyl” and “hydroxy” refer to the —OH group.

The term “hydroxyalkyl” refers to an alkyl group substituted with an —OH group.

The term “mercapto” or “thio” refers to the —SH group. The term “silyl” refers to groups comprising silicon atoms (Si).

As used herein the term “alkoxysilane” refers to a compound comprising one, two, three, or four alkoxy groups bonded to a silicon atom. For example, tetraalkoxysilane refers to Si(OR)₄, wherein R is alkyl. Each alkyl group may be the same or different. An “alkylsilane” refers to an alkoxysilane wherein one or more of the alkoxy groups has been replaced with an alkyl group. Thus, an alkylsilane comprises at least one alkyl-Si bond. The term “fluorinated silane” refers to an alkylsilane wherein one of the alkyl groups is substituted with one or more fluorine atoms. The term “cationic or anionic silane” refers to an alkylsilane wherein one of the alkyl groups is further substituted with an alkyl substituent that has a positive (i.e., cationic) or a negative (i.e. anionic) charge, or may become charged (i.e., is ionizable) in a particular environment (i.e., in vivo).

The term “silanol” refers to the Si—OH group.

The embodiments described in one aspect of the present invention are not limited to the aspect described. The embodiments may also be applied to a different aspect of the invention as long as the embodiments do not prevent these aspects of the invention from operating for its intended purpose.

I. Oral Care Composition Comprising Nitric Oxide Releasing Particle

The present invention provides an oral care composition comprising a nitric oxide releasing particle and an orally-acceptable carrier. In some embodiments, the nitric oxide releasing article comprises at least one nitric oxide donor. In another embodiment, the nitric oxide releasing particle comprises at least one nitric oxide donor and a co-condensed silica network. In another embodiment, the nitric oxide releasing particle comprises a nitric oxide donor; an interior region having a volume, the volume of the interior region at least partially filled by a core comprising a co-condensed silica network, and an exterior region.

As used herein, the terms “nitric oxide donor” or “NO donor” refer to species that donate, generate, release, and/or directly or indirectly transfer a nitric oxide species, and/or stimulate the endogenous production of nitric oxide in vivo and/or elevate endogenous levels of nitric oxide in vivo such that the biological activity of the nitric oxide species is expressed at the intended site of action.

As used herein, the terms “nitric oxide releasing” or “nitric oxide donating” refer to methods of donating, releasing and/or directly or indirectly transferring any of the three redox forms of nitrogen monoxide (NO⁺, NO⁻, NO). In some cases, the nitric oxide releasing or donating is accomplished such that the biological activity of the nitrogen monoxide species is expressed at the intended site of action.

As used herein, the term “particle” is not limited to particle with a uniformed size. It also refers to an amorphous solid, colloidal particle, a film or coating with various shape, thickness and dimension, or any particles with suitable size or shape.

In the present invention, the nitric oxide releasing particles may be prepared by methods described in international publication no. WO 2006/128121, the disclosure of which is incorporated by reference in its entirety.

In some embodiments, the nitric oxide donor is diazeniumdiolate and the nitric oxide donor is formed from an aminoalkoxysilane by a pre-charging method such that diazeniumdiolated aminoalkoxysilane is formed, and the co-condensed silica network is synthesized from the condensation of a silane mixture comprising an alkoxysilane and the diazeniumdiolated aminoalkoxysilane to form a nitric oxide donor modified co-condensed silica network. According to some embodiments of the invention, the co-condensed silica network is prepared according to a modified Stöber synthesis. An exemplary procedure of preparing co-condensed silica network is further detailed in Example I.

The co-condensed silica network may be silica particles with a uniformed size, a collection of silica particles with a variety of size, amorphous silica, a fumed silica, a nanocrystalline silica, ceramic silica, colloidal silica, a silica coating, a silica film, organically modified silica, mesoporous silica, silica gel, bioactive glass, or any suitable form or state of silica.

As used herein, the “pre-charging method” means that aminoalkoxysilane is “pretreated” or “precharged” with nitric oxide prior to the co-condensation with alkoxysilane. In some embodiments, the precharging nitric oxide may be accomplished by chemical methods. In another embodiment, the “pre-charging” method may be used to create co-condensed silica networks and materials more densely functionalized with NO-donors. In some embodiments, the amino group of aminoalkoxysilane is substituted with a diazeniumdiolate to form a diazeniumdiolated aminoalkoxysilane nitric oxide donor.

In some embodiments, the nitric oxide donor is selected from the diazeniumdiolate, nitrosamine, hydroxyl nitrosamine, nitrosothiol, hydroxyl amine, hydroxyurea, and a combination thereof. In other embodiments, the nitric oxide donor is an N-diazeniumdiolate.

In some embodiments, the alkoxysilane is a tetraalkoxysilane having the formula Si(OR)₄, wherein R is an alkyl group. The R groups may be the same or different. In some embodiments the tetraalkoxysilane is selected tetramethyl orthosilicate (TMOS) or tetraethyl orthosilicate (TEOS). In some embodiments, the aminoalkoxysilane has the formula: R″—(NH—R′)ₙ—Si(OR)₃, wherein R is alkyl, R′ is alkylene, branched alkylene, or aralkylene, n is 1 or 2, and R″ is selected from alkyl, cycloalkyl, aryl, and alkylamine.

In some embodiments, the aminoalkoxysilane may be selected from N-(6-aminohexyl)aminopropyltrimethoxysilane (AHAP3); N-(6-aminoethyl)aminopropyltrimethoxysilane; (3-trimethoxysiiylpropyl)di-ethylenetriamine (DET3); (aminoethylaminomethyl)phenethyltrimethoxysilane (AEMP3); [3-(methylamino)propyl]trimethoxysilane; N-butylamino-propyltrimethoxysilane; N-ethylaminoisobutyltrimethoxysilane; N-phenylamino-propyltrimethoxysilane; and N-cyclohexylaminopropyltrimethoxysilane.

In some embodiments, the aminoalkoxysilane has the formula: NH[R′—Si(OR)₃]₂, wherein R is alkyl and R′ is alkylene. In some embodiments, the aminoalkoxysilane may be selected from bis-[3-(trimethoxysilyl)propyl]amine and bis-[(3-trimethoxysilyl)propyl]ethylenediamine.

In some embodiments, as described herein above, the aminoalkoxysilane is precharged for NO-release and the amino group is substituted with a diazeniumdiolate. Therefore, in some embodiments, the diazeniumdiolated aminoalkoxysilane has the formula: R″—N(NONO⁻X⁺)—R′—Si(OR)₃, wherein R is alkyl, R′ is alkylene or aralkylene, R″ is alkyl or alkylamine, and X⁺ is a cation selected from Na⁺, K⁺ and Li⁺.

The composition of the silica network, (e.g., amount or the chemical composition of the aminoalkoxysilane) and the nitric oxide charging conditions (e.g., the solvent and base) may be varied to optimize the amount and duration of nitric oxide release. Thus, in some embodiments, the composition of the presently disclosed silica particles may be modified to regulate the half-life of NO release from silica particles.

The above definition of alkoxysilane and aminoalkoxysilane are not limited to oral care composition, and it may also be applied to other aspects of the present invention such as devices for oral care, polymer compositions and methods of providing oral health benefits.

In some embodiments, the oral composition is in a form of toothpaste, a gel, powder, a solution, a fiber, a suspension, an emulsion, a lozenge, a mucoadhesive vehicle, a tablet or a gum.

In some embodiments, the nitric oxide releasing particle is in the form of a nanoparticle or a microparticle. In some embodiments, the nitrogen oxide releasing particle has a diameter in a range of about 2 nm to about 10 μm. In other embodiments, the nitrogen oxide releasing particle has an average diameter in a range of about 10 um to about 30 μm.

In some embodiments, the orally acceptable carrier is an organic polymer. In other embodiments, orally acceptable carrier is toothpaste, mouth wash or a dental floss.

As used herein, “incorporate” means that the nitric oxide releasing particles may either crosslink to or covalently bond to, but not limited to, a polymer, a reagent, a device, a dye, or a pigment.

In some embodiments, the oral composition further comprises at least one therapeutic agent. In other embodiments, the nitric oxide releasing particles are incorporated into the therapeutic agent. In another embodiment, the therapeutic agent is selected from anti-cancer therapeutics, antimicrobial agents, pain relievers, anti-inflammatories, vasodialators and immune-suppresants for use in treating oral care disease states.

The combination of nitric oxide and additional therapeutic agents may also be applied in other aspects of the invention such as devices for oral care and methods of providing oral health benefits.

Additional therapeutic agents may be incorporated into the particles themselves or be part of a formulation comprising the particles or doses as a separate formulation prior to, after, or at the same time as a formulation including the particles. The additional agents include, but are not limited to, anti-cancer therapeutics, anti-microbial agents, pain relievers, anti-inflammatories, vasodialators, and immune-suppresants, as well as any other known therapeutic agents that may enhance the alleviation of the disease or condition being treated. In some embodiments of the present invention, wherein the additional therapeutic agent or agents are incorporated into the NO-releasing particles, the additional therapeutic agent may be associated with any of the exterior, the interior or the core of the silica network. For example, the additional agents may be encapsulated into the core or linkers in the interior portion of the silica network. The additional agents may also be covalently attached to the core, the interior or the exterior of the silica network. The choice of additional therapeutic agents to be used in combination with an NO-releasing particle will depend on various factors including, but not limited to, the type of disease, the age, and the general health of the subject, the aggressiveness of disease progression, and the ability of the subject to tolerate the agents that comprise the combination.

A variety of chemical compounds, also described as “antineoplastic” agents or “chemotherapeutic agents” may be used in combination with or incorporated into the presently disclosed NO-releasing particles used in the treatment of cancers related to the oral cavity.

Such chemotherapeutic compounds include, but are not limited to, alkylating agents, DNA intercalators, protein synthesis inhibitors, inhibitors of DNA or RNA synthesis, DNA base analogs, topoisomerase inhibitors, anti-angiogenesis agents, and telomerase inhibitors or telomeric DNA binding compounds. For example, suitable alkylating agents include, but are not limited to, alkyl sulfonates, such as busulfan, improsulfan, and piposulfan; aziridines, such as a benzodizepa, carboquone, meturedepa, and uredepa; ethylenimines and methylmelamines, such as altretamine, triethylenemelamine, triethylenephosphoramide, triethylenethiophosphoramide, and trimethylolmelamine; nitrogen mustards such as chlorambucil, chlornaphazine, cyclophosphamide, estramustine, iphosphamide, mechlorethamine, mechlorethamine oxide hydrochloride, melphalan, novembichine, phenesterine, prednimustine, trofosfamide, and uracil mustard; nitroso ureas, such as carmustine, chlorozotocin, fotemustine, lomustine, nimustine, and ranimustine.

Antibiotics used in the treatment of cancer include, but are not limited to, dactinomycin, daunorubicin, doxorubicin, idarubicin, bleomycin sulfate, mytomycin, plicamycin, and streptozocin. Chemotherapeutic antimetabolites include, but are not limited to, mercaptopurine, thioguanine, cladribine, fludarabine phosphate, fluorouracil (5-FU), floxuridine, cytarabine, pentostatin, methotrexate, and azathioprine, acyclovir, adenine β-1-D-arabinoside, amethopterin, aminopterin, 2-aminopurine, aphidicolin, 8-azaguanine, azaserine, 6-azauracil, 2′-azido-2′-deoxynucleosides, 5-bromodeoxycytidine, cytosine-1-D-arabinoside, diazooxynorleucine, dideoxynucleosides, 5-fluorodeoxycytidine, 5-fluorodeoxyuridine, and hydroxyurea.

Chemotherapeutic protein synthesis inhibitors include, but are not limited to, abrin, aurintricarboxylic acid, chloramphenicol, colicin E3, cycloheximide, diphtheria toxin, edeine A, emetine, erythromycin, ethionine, fluoride, 5-fluorotryptophan, fusidic acid, guanylyl methylene diphosphonate and guanylyl imidodiphosphate, kanamycin, kasugamycin, kirromycin, and O-methyl threonine. Additional protein synthesis inhibitors include, but are not limited to, modeccin, neomycin, norvaline, pactamycin, paromomycine, puromycin, ricin, shiga toxin, showdomycin, sparsomycin, spectinomycin, streptomycin, tetracycline, thiostrepton, and trimethoprim. Inhibitors of DNA synthesis, including alkylating agents such as dimethyl sulfate, mitomycin C, nitrogen and sulfur mustards, intercalating agents, such as acridine dyes, actinomycins, adriamycin, anthracenes, benzopyrene, ethidium bromide, propidium diiodide-intertwining, and agents, such as distamycin and netropsin, may be used as part of the presently disclosed cancer treatments. Topoisomerase inhibitors, such as coumermycin, nalidixic acid, novobiocin, and oxolinic acid, inhibitors of cell division, including colcemide, colchicine, vinblastine, and vincristine; and RNA synthesis inhibitors including actinomycin D, σ-amanitine and other fungal amatoxins, cordycepin (3′-deoxyadenosine), dichlororibofuranosyl benzimidazole, rifampicine, streptovaricin, and streptolydigin also may be combined with or incorporated into the particles of the presently disclosed subject matter to provide a suitable cancer treatment.

Thus, current chemotherapeutic agents that may be used as part of or in combination with the presently describe NO-releasing particles include, but are not limited to, adrimycin, 5-fluorouracil (5FU), etoposide, camptothecin, actinomycin-D, mitomycin, cisplatin, hydrogen peroxide, carboplatin, procarbazine, mechlorethamine, cyclophosphamide, ifosfamide, melphalan, chlorambucil, bisulfan, nitrosurea, dactinomycin, duanorubicin, doxorubicin, bleomycin, plicomycin, tamoxifen, taxol, transplatimun, vinblastin, and methotrexate, and the like.

As used herein, the term “antimicrobial agent” refers to any agent that kills, inhibits the growth of, or prevents the growth of a bacteria, fungus, yeast, or virus. Suitable antimicrobial agents that may be incorporated into the presently disclosed NO-releasing particles to aid in the treatment or prevention of a microbial infection, include, but are not limited to, antibiotics such as vancomycin, bleomycin, pentostatin, mitoxantrone, mitomycin, dactinomycin, plicamycin and amikacin. Other antimicrobial agents include, but are not limited to, antibacterial agents such as 2-p-sulfanilyanilinoethanol, 4,4′-sulfinyldianiline, 4-sulfanilamidosalicylic acid, acediasulfone, acetosulfone, amikacin, amoxicillin, amphotericin B, ampicillin, apalcillin, apicycline, apramycin, arbekacin, aspoxicillin, azidamfenicol, azithromycin, aztreonam, bacitracin, bambermycin(s), biapenem, brodimoprim, butirosin, capreomycin, carbenicillin, carbomycin, carumonam, cefadroxil, cefamandole, cefatrizine, cefbuperazonθ, cefclidin, cefdinir, cefditoren, cefepime, cefetamet, cefixime, cefmenoxime, cefininox, cefodizime, cefonicid, cefoperazone, ceforanide, cefotaxime, cefotetan, cefotiam, cefozopran, cefpimizole, cefpiramide, cefpirome, cefprozil, cefroxadine, ceftazidime, cefteram, ceftibuten, ceftriaxone, cefuzonam, cephalexin, cephaloglycin, cephalosporin C, cephradine, chloramphenicol, chlortetracycline, ciprofloxacin, clarithromycin, clinafloxacin, clindamycin, clindamycin phosphate, clomocycline, colistin, cyclacillin, dapsone, demeclocycline, diathymosulfone, dibekacin, dihydrostreptomycin, dirithromycin, doxycycline, enoxacin, enviomycin, epicillin, erythromycin, flomoxef, fortimicin(s), gentamicin(s), glucosulfone solasulfone, gramicidin S, gramicidin(s), grepafloxacin, guamecycline, hetacillin, imipenem, isepamicin, josamycin, kanamycin(s), leucomycin(s), lincomycin, lomefloxacin, lucensomycin, lymecycline, meclocycline, meropenem, methacycline, micronomicin, midecamycin(s), minocycline, moxalactam, mupirocin, nadifloxacin, natamycin, neomycin, netilmicin, norfloxacin, oleandomycin, oxytetracycline, p-sulfanilylbenzylamine, panipenem, paromomycin, pazufloxacin, penicillin N, pipacycline, pipemidic acid, polymyxin, primycin, quinacillin, ribostamycin, rifamide, rifampin, rifamycin SV, rifapentine, rifaximin, ristocetin, ritipenem, rokitamycin, rolitetracycline, rosaramycin, roxithromycin, salazosulfadimidine, sancycline, sisomicin, sparfloxacin, spectinomycin, spiramycin, streptomycin, succisulfone, sulfachrysoidine, sulfaloxic acid, sulfamidochrysoidine, sulfanilic acid, sulfoxone, teicoplanin, temafloxacin, temocillin, tetracycline, tetroxoprim, thiamphenicol, thiazolsulfone, thiostrepton, ticarcillin, tigemonam, tobramycin, tosufloxacin, trimethoprim, trospectomycin, trovafloxacin, tuberactinomycin and vancomycin. Exemplary antimicrobial agents may also include, but are not limited to, anti-fungals, such as amphotericin B, azaserine, candicidin(s), chlorphenesin, dermostatin(s), filipin, fungichromin, mepartricin, nystatin, oligomycin(s), perimycin A, tubercidin, imidazoles, triazoles, and griesofulvin.

In some embodiments, the antimicrobial agent is selected from NaF, SnF₂, sodium monofluorophosphate, triclosan, tinosan SDC, cetylpyridinium chloride, chlorhexidine, zinc citrate and alcohol.

In another embodiment, the co-condensed silica network of the oral composition is synthesized from the condensation of a silane mixture comprising an alkoxysilane and an aminoalkoxysilane, and the NO donor is formed via a post-charging method, and the nitric oxide donor is formed on an existing particle scaffold. In some embodiments, the existing particle scaffold comprises at least one amine group immobilized on at least one solid support resin. In one embodiment, the solid support resin comprises at least one composition selected from polystyrene, polyacrylate, polyvinylpyrolidone, co-condensed silica, or a combination thereof. Exemplary polystyrene resins include, but are not limited to, Tris-(a-aminoethyl)amine resin, PL-DETA resin, PL-EDA resin, PL-PPZ resin with loading ratios ranging from about 0.5 to about 6 mmol amine per gram of resin and sizes ranging from about 30 to about 300 μm. Exemplary amines on the solid support resin include, but are not limited to, primary amines, secondary amine, or cyclic secondary amines. In some embodiments, the co-condensed silica or silica network of the oral composition comprises cyclic azasilane/hexamethyldisilazane treated silicon dioxide. Cyclic azasilane/hexamethyldisilazane treated silicon dioxide is amorphous silica particles with ultimate particle size of 20 nm and they are commercially available from Gelest, Inc. It contains secondary amine, which may be used to store nitric oxide via the post-charging method.

II. Devices for Oral Care Comprising Nitric Oxide Releasing Particle

Another aspect of the present invention provides a device for oral care comprising a nitric oxide releasing particle which comprises at least one nitric oxide donor. The device is configured to expose a targeted site in an oral cavity of a subject to nitric oxide. In some embodiments, the nitric oxide releasing particle comprises at least one nitric oxide donor and a co-condensed silica network. In another embodiment, the nitric oxide releasing particle may comprise a nitric oxide donor; an interior region having a volume, the volume of the interior region at least partially filled by a core comprising a co-condensed silica network; and an exterior region.

The “patient” or “subject” treated in the many embodiments disclosed herein is a human patient, although it is to be understood that the principles of the presently disclosed subject matter indicate that the presently disclosed subject matter is effective with respect to all vertebrate species, including mammals, which are intended to be included in the terms “subject” and “patient.” In this context, a mammal is understood to include any mammalian species in which treatment is desirable, particularly agricultural and domestic mammalian species, such as horses, cows, pigs, dogs, and cats.

In some embodiments, the device is in a form of a mouthguard, a dental sealant, a medical device, a dental implant, a tray, a strip, a syringe, a cover, a pad/patch, a film, a sponge, a cream, a fiber, or a gel that is adapted to be applied on the targeted site. As used herein, “film”, “strip”, “pad/patch” may be a variety of shape, thickness and multiple dimensions.

In some embodiments, the nitric oxide donor is selected from diazeniumdiolate, nitrosamine, hydroxyl nitrosamine, nitrosothiol, hydroxyl amine, hydroxyurea, and a combination thereof. In another embodiment, the nitric oxide donor is an N-diazeniumdiolate.

In some embodiments, the nitric oxide donor is diazeniumdiolate, and the nitric oxide donor is formed from an aminoalkoxysilane by a pre-charging method such that diazeniumdiolated aminoalkoxysilane is formed. Then, the co-condensed silica network is synthesized from the condensation of a silane mixture comprising an alkoxysilane and the diazeniumdiolated aminoalkoxysilane to form a nitric oxide donor modified co-condensed silica network. The diazeniumdiolated aminoalkoxysilane has a formula of R″—N(NONO⁻X⁺)—R′—Si(OR)₃, wherein: R is alky; R′ is alkylene or aralkylene; R″ is alkyl or alkylamine; and X⁺ is a cation selected from Na⁺ and K⁺.

In some embodiments of the device for oral care, the co-condensed silica network further comprises at least one crosslinkable functional moiety of formula (R₁)ₓ(R₂)_{y}SiR₃, wherein R₁ and R₂ is each independently C₁₋₅ alkyl or C₁₋₅ alkoxyl, X and Y is each independently 0, 1, 2, or 3, and X+Y equal to 3, and R₃ is a crosslinkable functional group. In a further embodiment, R₁ is C₁₋₃ alkoxyl, and R₂ is methyl. In another embodiment, R₃ is selected from acrylo, alkoxy, epoxy, hydroxy, mercapto, amino, isocyano, carboxy, vinyl and urea. R₃ imparts an additional functionality to the silica which results in a multifunctional device. Yet, in another embodiment, the crosslinkable functional moiety is selected from methacryloxymethyltrimethoxysilane, methacryloxypropyltrimethoxysilane, methacryloxypropyltriethoxysilane, 3-acryloxypropyl)trimethoxysilane, N-(3-methyacryloxy-2-hydroxypropyl)-3-aminopropyltriethoxysilane, 5,6-epoxyhexyltriethoxysilane, 2-(3,4-epoxycyclohexyl)ethyl trimethoxysilane, 3-glycidoxypropyl)trimethoxysilane, 3-aminopropyltrimethoxysilane, 3-aminopropyltriethoxysilane, 3-isocyanatopropyltriethoxysilane, 3-mercaptopropyltrimethoxysilane, mercaptopropyltriethoxysilane, 11-mercaptoundecyltrimethoxysilane, 2-cyanoethyltriethoxysilane, ureidopropyltriethoxysilane, ureidopropyltrimethoxysilane, vinylmethyldiethoxysilane, vinylmethyldimethoxysilane, vinyltriethoxysilane, vinyltrimethoxysilane, vinyltriisopropoxysilane and vinyltris(2-methoxyethoxy)silane. In some embodiments, R₃ may be used to cross-link the NO-donor modified silica with or within polymeric matrices.

In some embodiments, the nitric oxide donor modified co-condensed silica network is incorporated into an organic polymer to form a nitric oxide incorporated polymer. In another embodiment, the device is coated with the nitric oxide incorporated polymer. In another embodiment, the device is at least partially made of the nitric oxide incorporated polymer. Such incorporation may be accomplished through physically embedding the particles into polymer surfaces, via electrostatic association of particles onto polymeric surfaces, or by covalent attachment or cross-linking of particles onto reactive groups on the surface of a polymer. Alternatively, the particles may be mixed into a solution of liquid polymer precursor, becoming entrapped in the polymer matrix when the polymer is cured. Polymerizable groups may also be used to functionalize the exterior of the particles, whereupon, the particles may be co-polymerized into a polymer during the polymerization process. Suitable polymers into which the NO-releasing particles may be incorporated include, but are not limited to, polyolefins, such as polystyrene, polypropylene, polyethylene, polytetrafluoroethylene, and polyvinylidene, as well as polyacrylates, polyesters, polyethers, polyurethanes, and the like. In some embodiments, the organic polymer is selected from cellulose, polyacrylate, polyamide, polycarbonate, polyester, poly (ether ether ketone), polyethylene, poly(ethylene glycol), poly(ethylene terephthalate), polyimide, polytetrafluoroethylene, polyurethane, polyvinylchloride, polyvinylpyrrolidone, polystyrene and polysiloxane. In a particular embodiment, polyurethanes may include medically segmented polyurethanes.

In some embodiments, the release of nitric oxide is triggered by contacting the nitric oxide releasing particle with a proton donor. The amount and rate of nitric oxide released may be controlled by adjusting the pH of the environment of the nitric oxide releasing particle. In some embodiments, in order to release the nitric oxide, the pH of the environment of the nitric oxide releasing particle is adjusted to a range of about 3 to about 8. In another environment, the pH of the environment of the nitric oxide releasing particle is adjusted to a range of about 3.5 to about 7.0.

In some embodiments, the device comprises at least two separate phases, wherein at least one phase comprises the nitric oxide releasing particle, and the device is configured to provide admixing upon administration. In another embodiment, the admixing triggers the release of nitric oxide. In some embodiments, the release of nitric oxide is triggered by change of pH of the phases containing nitric oxide donor upon admixing of two phases. One example is that two phases comprise solutions of different pH. Upon admixing two phases, the resulting pH in the phase containing nitric oxide provides a desired kinetics of nitric oxide release. In some embodiments, in order to release the nitric oxide, the pH of the phase after mixing is adjusted to a range of about 3 to about 8. In another environment, the pH of the phase after mixing is adjusted to a range of about 3.5 to about 7.0. The advantages of device of delivery nitric oxide through admixing different phases are the rate and/or concentrations of nitric oxide may be adjusted to provide an optimal therapeutic dose of nitric oxide for different oral diseases.

In some embodiments, the phase comprising the nitric oxide releasing particles comprises a solvent selected from glycerol, propylene glycol, dipropylene glycol, ethoxydiglycol, butylene glycol, dimethyl isosorbide, triethylcitrate, methanol, ethanol, propanol, butanol, alcohol, a basic aqueous solution and a combination thereof. In another embodiment, the phase without the nitric oxide releasing particle comprises a proton donor. Yet, in other embodiments, the proton donor is selected from water, oxonium ion, ammonium ion, lactic acid, acetic acid, caprylic acid, lauric acid, palmitic acid, stearic acid, salicylic acid, malonic acid, malic acid, fumaric acid, adipic acid, succinic acid, phosphoric acid, glucuronic acid, citric acid, ascorbic acid and an amino acid.

In some embodiments, the release of nitric oxide is triggered by exposing the nitric oxide releasing particles to the moisture in the oral cavity. As used herein, “moisture” in the mouth includes, but is not limited to, saliva or any solution contained in the mouth such as water, juice, mouth wash. In some embodiments, the moisture is saliva. In some embodiments, the release of nitric oxide is triggered by exposing the nitric oxide releasing particles to the temperature in the oral cavity.

In some embodiments, the nitric oxide is enclosed by a polymeric membrane with at least one opening, and the opening of the polymeric membrane enlarges upon exposure to moisture or temperature in the oral cavity to release nitric oxide releasing particles. For example, the polymer membrane is configured to swell upon exposure to saliva or moisture in the oral cavity, and the pore sizes of the polymer membrane enlarge to release the nitric oxide releasing particles.

In another embodiment, the co-condensed silica network of the device is synthesized from the condensation of a silane mixture comprising an alkoxysilane and an aminoalkoxysilane, and the nitric oxide donor is formed by a post-charging method, wherein the nitric oxide donor is formed on an existing particle scaffold. In some embodiments, the existing particle scaffold comprises at least one amine group immobilized on at least one solid support resin. In one embodiment, the solid support resin comprises at least one composition selected from polystyrene, polyacrylate, polyvinylpyrolidone, co-condensed silica, or a combination thereof. Exemplary polystyrene resins include, but are not limited to, Tris-(a-aminoethyl)amine resin, PL-DETA resin, PL-EDA resin, PL-PPZ resin with loading ratios ranging from about 0.5 to about 6 mmol amine per gram of resin and sizes ranging from about 30 to about 300 μm. Exemplary amines on the solid support resin include, but are not limited to, primary amines, secondary amine, or cyclic secondary amines. In some embodiments, the co-condensed silica or silica network of the device comprises cyclic azasilane/hexamethyldisilazane treated silicon dioxide.

In some embodiments, the device according to the invention may be used in any post or pre-surgery treatment to prevent, treat, and/or alleviate any kind of infection or inflammation. In some embodiments, the device is used to prevent disorders post surgery in the oral cavity. The effects of nitric oxide may be anti-inflammatory, anti-pathogenic, anti-viral and/or anti-bacterial.

III. Polymer Composition for Oral Care Comprising Nitric Oxide Releasing Particles

Another aspect of the present invention provides a polymeric composition for oral care comprising an organic polymer and a nitric oxide-releasing particle, which comprises at least one nitric oxide donor. In some embodiments, the nitric oxide releasing particle comprises at least one nitric oxide donor and a co-condensed silica network. In another embodiment, the nitric oxide releasing particle comprises at least one nitric oxide donor and an interior region having a volume, the volume of the interior region at least partially filled by a core comprising a co-condensed silica network; and an exterior region. In some embodiments, the nitric oxide releasing particle is cross-linked to the organic polymer. For example, the nitric oxide releasing particle may covalently bond to the backbone of the polymer chain or side chains of the polymer.

IV. Methods of Providing Oral Health Benefits

Another aspect of the present invention provides methods of providing one or more oral health benefits to a subject. The methods comprise contacting an effective amount of an oral care composition in an oral cavity of the subject. The oral care composition comprises nitric oxide releasing particle and an orally-acceptable carrier and the nitric oxide releasing particle comprises at least one nitric oxide donor. In some embodiments, the nitric oxide releasing particle comprises a nitric oxide donor and a co-condensed silica network. In another embodiment, the nitric oxide releasing particle comprises a nitric oxide donor; an interior region having a volume, the volume of the interior region at least partially filled by a core comprising a co-condensed silica network; and an exterior region.

A further aspect of the present invention provides a method of providing one or more oral health benefits to a subject comprising exposing a targeted site in an oral cavity of the subject to a device described herein.

In some embodiments, the above methods provide treatment and/or prevention of infection, such as infection caused by bacteria, viruses, fungi or yeast or herpes. In some embodiments, the above methods provide treatment and/or prevention of dental carries, plaque formation and accumulation, gingivitis, periodontitis disease or other stomatognathic diseases. For example, the oral composition or device of the present invention may be used by dentists as a post surgery treatment to prevent the immediate plaque formation which begins within hours after the process. Another example is that the nitric oxide releasing particles may be incorporated into dental sealants to prevent bacteria adhesion and plaque accumulation. For example, nitric oxide releasing silica may be incorporated into dental implants used to fill voids or empty spaces between teeth and the gum line.

Yet, in another embodiment, in the method discussed above, the amount and rate of the release of nitric oxide is regulated by adjusting pH of the oral cavity or light exposed to the nitric oxide particles. For example, N-diazeniumdiolates, a class of nitric oxide donors, are subject to a proton initiated releasing mechanism. The release of nitric oxide from N-diazeniumdiolate may be facilitated by lowering the pH to acidic conditions. On the other hand, at a neutral pH, the release of nitric oxide may be triggered, but the half-lives of nitric oxide release are shortened. Alternatively, at a basic pH, the stability of the nitric oxide donor is enhanced and the storage/lifetime of the nitric oxide donor is increased. In other embodiments, the amount and rate of the release of nitric oxide is facilitated by decreasing pH.

A representative example of release of nitric oxide triggered by light is nitrosothiol type nitric oxide donor. The nitrosothiols may undergo homolytic cleavage when they are exposed to broad spectrum of white light or to specific wavelengths targeting their individual absorption maxima.

The advantages of the present invention are that nitric oxide may be delivered in a controlled and targeted manner. In particular, for oral care applications, nitric oxide may be released as a gas to diffuse through plaque biofilms and kill bacterial beneath.

### EXAMPLES

### I. Preparation of Nitric Oxide Loaded Silica

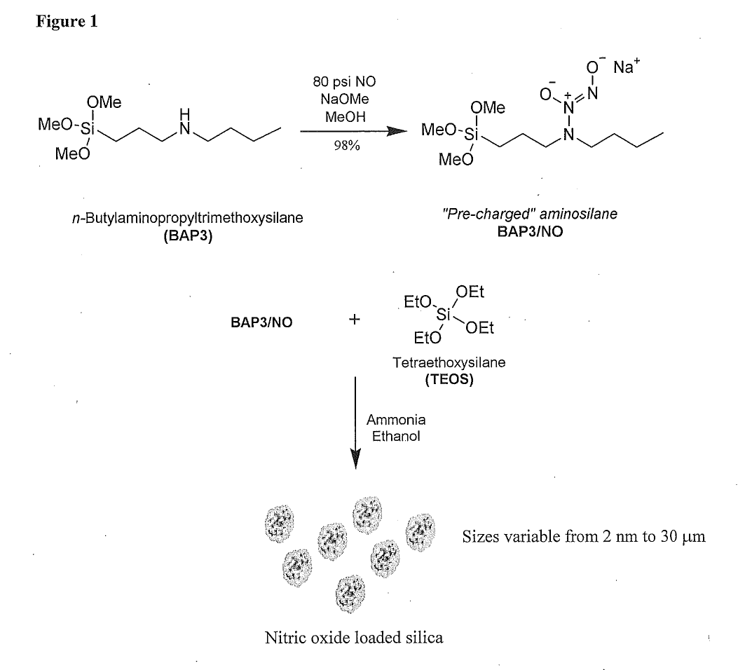

An exemplary preparation of nitric oxide loaded silica is shown in FIG. 1. The nitric oxide loaded silica is prepared by co-condensation of an alkoxysilane and pre-charged aminoalkoxysilane according to a modified Stöber synthesis. The variation of the reaction condition is within the knowledge of one of ordinary skill in the art.

### II. Preparation of Multifunctional Nitric Oxide Loaded Silica

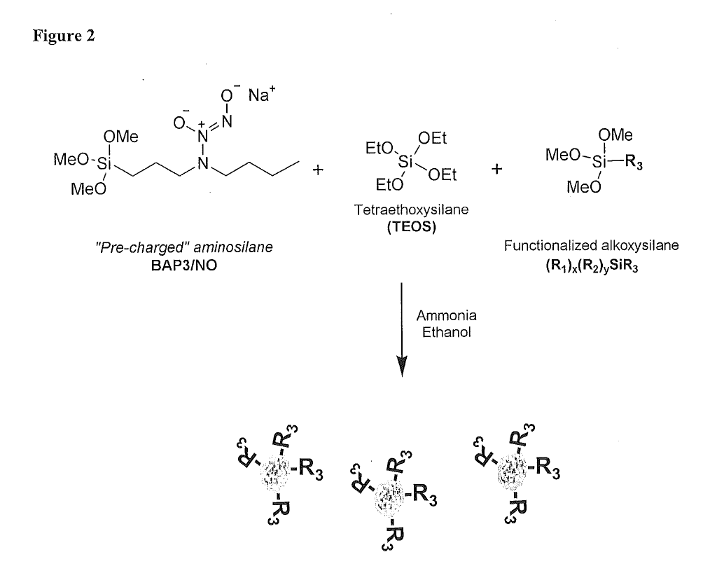

An exemplary preparation of multifunctional nitric oxide loaded silica is shown in FIG. 2. The multifunctional nitric oxide loaded silica is prepared via the co-condensation of an alkoxysilane, a pre-charged aminoalkoxysilane, and a silane that comprises at least one crosslinkable functional moiety according to a modified Stöber synthesis. The variation of the reaction condition is within the knowledge of one of ordinary skill in the art.

III. Preparation of Antimicrobial Nitric Oxide Releasing Particle

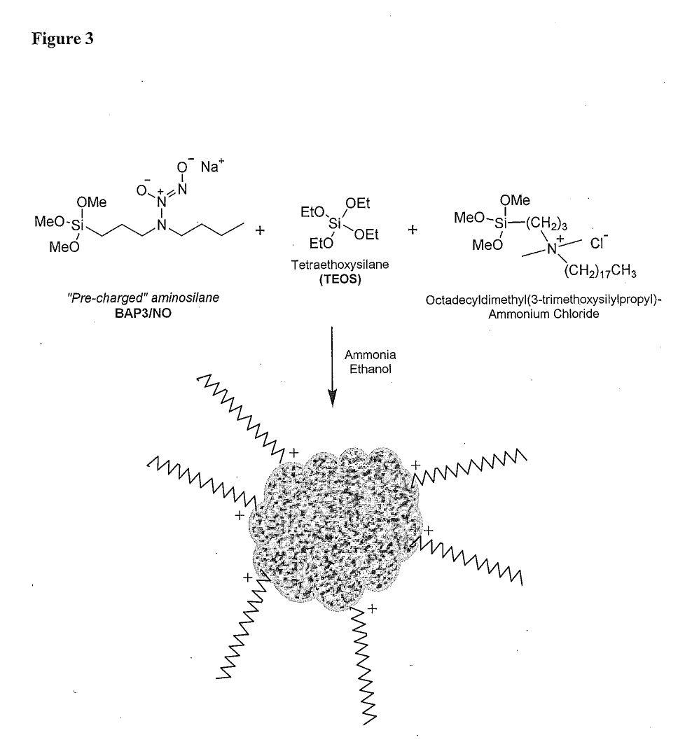

An exemplary preparation of antimicrobial nitric oxide releasing particle is shown in FIG. 3. The enhanced antimicrobial nitric oxide releasing silica particle is prepared via the co-condensation of an alkoxysilane, a pre-charged aminoalkoxysilane, and a silane that comprises an antimicrobial agent covalently linked to the third alkoxysilane. The variation of the reaction condition is within the knowledge of one of ordinary skill in the art.

IV. Methods of Incorporating Nitric Oxide Releasing Silica into a Polymeric Matrix

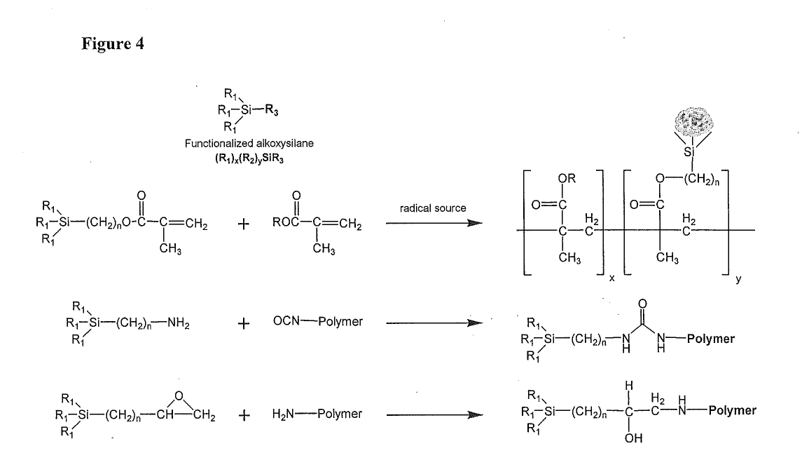

Some exemplary methods of incorporating nitric oxide releasing silica into a polymeric matrix are shown in FIG. 4. The exemplary methods use a functionalized alkoxysilane which comprises at least one crosslinkable residue. FIG. 4 demonstrates that several synthetic methods may be used to prepare poly(acrylate) polymers, urethanes, or fillers added to a polymer matric through epoxy coupling. The reactive groups on the polymer matrix may be located on the polymer backbone, sidechains, or in the form of prepolymers.

### V. Experiment of the Antibacterial Effect of Nitric Oxide Releasing Particles

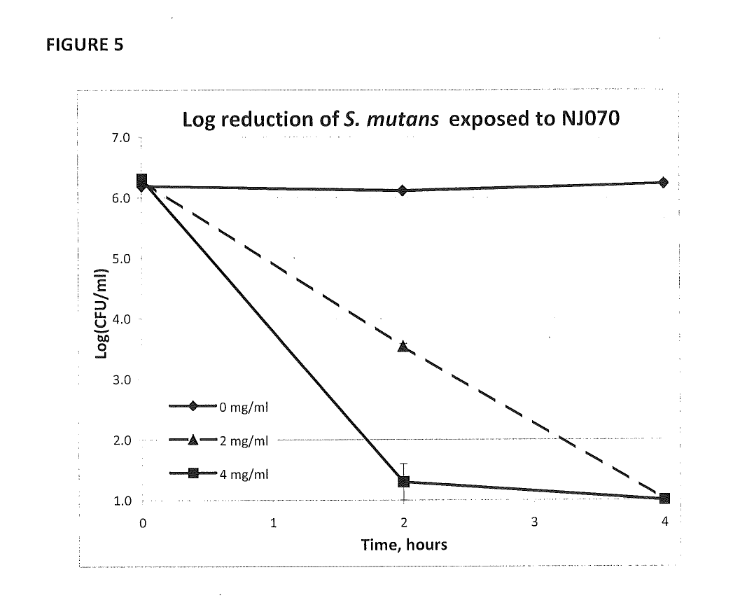

An exemplary time-based killing experiment of Streptococcus mutans (ATCC 25175) conducted with NO-releasing silica composition NJ070 prepared according to the general procedure outlined in Example I. At 10⁶ innoculum of S. mutans is challenged with various doses of NJ070 and resulted in >5 log reduction in CFU/mL for both 2 mg/mL and 4 mg/mL concentrations at 4 hours. FIG. 5 graphically demonstrates the log reduction in CFU/mL of Streptococcus mutans, a gram positive bacterium commonly found in the oral cavity when exposed to nitric oxide releasing co-condensed silica particles at various doses.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. A device comprising at least two separate phases, wherein a first phase comprises a nitric oxide releasing particle comprising a diazeniumdiolate and a second phase regulates the release of nitric oxide, and wherein the device is configured to admix the two separate phases upon administration.

2. The device of claim 1, wherein the at least two separate phases each comprise a solution.

3. The device of claim 2, wherein the solution of each phase is a different pH.

4. The device of claim 1, wherein the admixing triggers the release of nitric oxide.

5. The device of claim 1, wherein the admixing adjusts the pH of the first phase to facilitate the release of nitric oxide.

6. The device of claim 5, wherein the pH of the first phase is adjusted to a pH from about 3 to about 8.

7. The device of claim 1, wherein decreasing the pH of the first phase facilitates the amount and rate of release of nitric oxide.

8. The device of claim 1, wherein prior to admixing the first phase has a basic pH.

9. The device of claim 8, wherein the stability of the diazeniumdiolate in the first phase is enhanced compared to a diazeniumdiolate in a phase having a neutral pH.

10. The device of claim 8, wherein the storage/lifetime of the diazeniumdiolate in the first phase is increased compared to a diazeniumdiolate in a phase having a neutral pH.

11. The device of claim 1, wherein the at least two separate phases have a different pH and upon admixing the resulting pH provides a desired kinetics of nitric oxide release.

12. The device of claim 1, wherein the first phase comprises a solvent selected from the group consisting of glycerol, propylene glycol, dipropylene glycol, ethoxydiglycol, butylene glycol, dimethyl isosorbide, triethylcitrate, methanol, ethanol, propanol, butanol, alcohol, a basic aqueous solution, and any combination thereof.

13. The device of claim 1, wherein the second phase comprises a proton donor.

14. The device of claim 13, wherein the proton donor is selected from the group consisting of water, oxonium ion, ammonium ion, lactic acid, acetic acid, caprylic acid, lauric acid, palmitic acid, stearic acid, salicylic acid, malonic acid, malic acid, fumaric acid, adipic acid, succinic acid, phosphoric acid, glucuronic acid, citric acid, ascorbic acid, an amino acid, and any combination thereof.

15. The device of claim 1, wherein the device comprises a mouthguard.

16. A device comprising at least two separate phases, wherein a first phase comprises a nitric oxide releasing particle comprising a diazeniumdiolate and has a basic pH and a second phase regulates the release of nitric oxide and has a pH that is different than the pH of the first phase, and wherein the device is configured to admix the two separate phases upon administration to provide a desired kinetics of nitric oxide release.

17. The device of claim 16, wherein the pH of the first phase is adjusted to a pH from about 3 to about 8 upon admixing of the first phase and second phase.

18. The device of claim 16, wherein the first phase comprises a solvent selected from the group consisting of glycerol, propylene glycol, dipropylene glycol, ethoxydiglycol, butylene glycol, dimethyl isosorbide, triethylcitrate, methanol, ethanol, propanol, butanol, alcohol, a basic aqueous solution, and any combination thereof.

19. The device of claim 16, wherein the second phase comprises a proton donor.

20. The device of claim 19, wherein the proton donor is selected from the group consisting of water, oxonium ion, ammonium ion, lactic acid, acetic acid, caprylic acid, lauric acid, palmitic acid, stearic acid, salicylic acid, malonic acid, malic acid, fumaric acid, adipic acid, succinic acid, phosphoric acid, glucuronic acid, citric acid, ascorbic acid, an amino acid, and any combination thereof.
